(19) United States
(12) Patent Application Publication
(10) Pub. No.: US 2018/0265910 A1
(43) Pub. Date: Sep. 20, 2018

(54) Cleavage of Fucose in N-Glycans

(71) Applicant: New England Biolabs, Inc., Ipswich, MA (US)

(72) Inventors: Christopher H. Taron, Essex, MA (US); Saulius Vainauskas, Newburyport, MA (US); Xiaofeng Shi, Beverly, MA (US)

(73) Assignee: New England Biolabs, Inc., Ipswich, MA (US)

(21) Appl. No.: 15/462,287

(22) Filed: Mar. 17, 2017

(51) Int. Cl. C12Q 1/40; C12Q 1/34

(52) U.S. Cl. CPC C12Q 1/40; C12Y 302/01051; C12Y 302/01018; C12Y 302/01022; C12Y 302/01023; G01N 2333/924; C12Y 305/01052; C12Q 1/34; G01N 2400/02; G01N 2440/38; C12Y 302/01096

(57) ABSTRACT

Provided herein is an α-fucosidase that can cleave a conjugate comprising an N-glycan and a label where the label is added by amine reactive chemistry. The α-fucosidase also has an accelerated reaction time using Schiff base labeled N-glycans compared with BKF. A reaction mix, enzyme mix and kit comprising the α-fucosidase are provided, as well as a method for analyzing glycoproteins. The α-fucosidase finds particular use in analyzing the N-glycans of therapeutic glycoproteins.

### BACKGROUND

The terms glycan and polysaccharide are defined by IUPAC as synonyms meaning “compounds consisting of a large number of monosaccharides linked glycosidically.” However, in practice the term glycan may also be used to refer to the carbohydrate portion of a glycoconjugate, such as a glycoprotein, glycolipid, or a proteoglycan, even if the carbohydrate is only an oligosaccharide.

Many secreted eukaryotic proteins possess post-translational carbohydrate modifications of certain asparagine residues (N-glycans). N-glycans become attached to proteins in the endoplasmic reticulum of eukaryotic cells on the nitrogen (N) in the side chain of asparagine in the sequon of a protein. The sequon is an Asn-X-Ser or Asn-X-Thr sequence, where X is any amino acid except proline. N-Glycans are commonly comprised of the sugars galactose, N-acetyl neuraminic acid, N-acetylglucosamine, fucose, and mannose but may also contain other sugars such as N-acetylgalactosamine and N-glycolylneuraminic acid.

Many classes of biologic drugs (e.g., antibodies, fusion proteins, growth factors, cytokines, etc.) are glycoproteins that possess N-glycans. The composition of these glycans can affect the stability, bioactivity, and serum half-life of a biologic drug. As such, glycan structure is designated a critical quality attribute (CQA) that must be monitored during biologic manufacturing, and the glycan profile of a finished product is used to assess the consistency of a manufacturing process from batch to batch.

To assess their composition, glycans can be structurally profiled using a variety of analytical methods including liquid chromatography, mass spectrometry, or capillary electrophoresis. Additionally, enzymes that remove sugars from the non-reducing end of glycans (exoglycosidases) can be used in concert with these analytical methods to sequence glycans and provide an orthogonal assessment of a glycan's structure.

The past decade has seen many advances in the structural analysis of N-glycans. In general, analytical workflows have significantly increased in speed, throughput and sensitivity. This progress has been driven by improvements to nearly every aspect of workflow design including instrument sensitivity, separations technologies, sample preparation and computational analysis of data. Within the past few years, sample preparation for N-glycan profiling has changed dramatically. Improved reagents and methods have simplified and shortened the process of releasing and fluorescently labeling N-glycans. For structural profiling, N-glycans are typically first removed from a peptide or protein using the enzyme PNGase F (New Engalnd Biolabs, Ipswich, MA). New formats of PNGase F have substantially improved the speed and completeness of N-glycan release from proteins (see for example US 2015/0346194). Following their release, N-glycans are then fluorescently labeled on their reducing end to enable their ultimate detection during downstream analyses. Recently, new chemistries for label attachment have given rise to a new generation of fluorescent labels that offer “instant” labeling of N-glycans and improved sensitivity in downstream analytical methods such as liquid chromatography, mass spectrometry, and capillary electrophoresis (see for example Cohen, et al; Anal Biochem. 1993 June;211(2):279-87; US2012/0107942; and WO2013/049622).

For decades, exoglycosidases have been used to assist in structural determination of glycans. These enzymes sequentially remove specific terminal sugars from oligosaccharides. Specific and complete removal of the targeted sugar by the exoglycosidase is critical in order to precisely characterize the structure of glycan. Arrays of exoglycosidases with various different specificities can be used to fully identify and order the sugars in N-glycan (and other glycans). The field has typically utilized a series of well-characterized and commercially available exoglycosidases for glycan characterization. These enzymes have performed well in the presence of traditional fluorescent labels that have been attached to N-glycans via Schiff-base chemistry although these reactions can be rather slow. There is, however, a problem with the performance of certain enzymes in the presence of fluorescent labels that have been attached to the glycosylamine at the reducing end of N-glycans via amine reactive chemistry (e.g. reactive carbamate chemistry). For example, one exoglycosidase commonly used in such assays, bovine kidney fucosidase, does not effectively cleave alpha 1,6 linked fucose from the core of N-glycans that have been labeled with this class of fluorescent labels. This problem is a major new hurdle for improved methods of enzyme-based structure verification of N-glycans. Another challenge has been performing a defucosylation reaction on a complex N-glycan attached to the glycoprotein or glycopeptide without having to denature the protein or first cleave at least in part, the N-glycan.

### SUMMARY

In general, a composition that is a reaction mix is provided that includes (a) an α-L-fucosidase having an amino acid sequence that is at least 90% identical to amino acids 23-359 of sequence of SEQ ID NO:1; and (b) a conjugate comprising an N-glycan and a label.

In one aspect, the label comprises a fluorophore and/or a charge tag. In various aspects of the reaction mix, the N-glycan of the conjugate comprises a core fucose; the N-glycan is linked to the label via the reducing end of the N-glycan; the N-glycan is from a therapeutic glycoprotein and/or the N-glycan is linked to the label via amine reactive chemistry or by a Schiff base condensation reaction and reduction.

In various aspects of the reaction mix, the conjugate is made by reacting an amine at the reducing end of an N-glycan with amine-reactive label; and/or the conjugate is the product of a reaction between an amine functionalized label and an aldehyde group at the reducing end of the N-glycan.

Other aspects of the reaction mix include in addition to the α1-6 fucosidase, one or more exoglycosidases selected from the group consisting of: α2-3 neuraminidase S, α2-3,6,8,9 neuraminidase A, α1-3,4,6 galactosidase, β1-4 galactosidase,β-N-acetylglucosaminidase S, and α1-2,3,6 mannosidase where the enzymes are combined in the same buffer. In another aspect, the reaction mix has a pH in the range of pH 3.0 to pH 5.5.

In general, an enzyme mix is provided that includes preferably in a single buffer (a) an α-fucosidase having an amino acid sequence that is at least 90% identical to amino acids 23-359 of sequence of SEQ ID NO:1; and (b) one or more additional exoglycosidases other than Omnitrophica exoglycosidases that do not naturally occur together in a cell and are preferably derived from multiple different cell sources. At least one exoglucosidase is preferably derived from a mammalian source. The exoglycosidase(s) may be selected from the group consisting of: α2-3 neuraminidase S, α2-3,6,8,9 neuraminidase A, α1-3,4,6 galactosidase, β1-4 galactosidase, β-N-acetylglucosaminidase S, and α1-2,3,6 mannosidase. In one aspect, none of the one or more exoglycosidases in the enzyme mix is glycosylated. In one aspect, the enzyme mix includes the one or more exoglycosidases selected from the group consisting of NAN1, ABS, CBG, SPG, GUH and JBM. In other aspects, the enzyme mix comprises two or more of the exoglycosidases; or three or more of the exoglycosidases. In one aspect, the enzyme mix comprises α2-3 neuraminidase S, α2-3,6,8,9 neuraminidase A, α1-3,4,6 galactosidase, β1-4 galactosidase and β-N-acetylglucosaminidase S.

In general, a kit is provided that includes (a) a α-fucosidase having an amino acid sequence that is at least 90% identical to amino acids 23-269 of sequence of SEQ ID NO:1; and (b) an amine-reactive label or amine functionalized label. In one aspect, the kit further includes PNGase F. In one aspect, the kit includes one or more exoglycosidases selected from the group consisting of: α2-3 neuraminidase S, α2-3,6,8,9 neuraminidase A, α1-3,4,6 galactosidase, β1-4 galactosidase, β-N-acetylglucosaminidase S, and α1-2,3,6 mannosidase. In one aspect, at least one of the one or more exoglycosidases is combined with the α1,6-fucosidase in a reaction mixture.

In general, a method is provided for cleaving fucose from an N-glycan, that includes (a) combining: (i) an α-fucosidase having an amino acid sequence that is at least 90% identical to amino acids 23-359 of sequence of SEQ ID NO:1 with (ii) a conjugate comprising an N-glycan and a label to make a reaction mix; and (b) incubating the reaction mix so as to cleave any core a1,6-fucose from the N-glycan. In one aspect, step (a) is done at a pH in the range of pH 3.0-pH 5.5. In another aspect, the method further includes: (c) detecting the cleaved glycan or the cleaved fucose after step (b). The detecting may be quantitative. It may be performed by liquid chromatography, mass spectrometry, capillary electrophoresis or any combination thereof. In one aspect, the method further comprises: cleaving a N-glycan from a glycoprotein; and conjugating the reducing end of the N-glycan with an amine-reactive label or an amine functionalized label to produce the conjugate comprising the N-glycan and label. For example, the cleaving may be done using PNGaseF. The protein may be a therapeutic glycoprotein.

In general, a method for cleaving fucose from an N-glycan, includes: (a) combining an α-fucosidase having an amino acid sequence that is at least 90% identical to amino acids 23-359 of sequence of SEQ ID NO:1 with an intact N-glycan linked glycoprotein or glycopeptide in a reaction mix; and (b) incubating the reaction mix so as to cleave any core α1,6-fucose from the N-glycan. In one aspect the glycoprotein is an antibody.

In general, a reaction mix is provided that includes: (a) an α-L-fucosidase having an amino acid sequence that is at least 90% identical to amino acids 23-359 of sequence of SEQ ID NO:1 ; and (b) a glycoprotein or glycopeptide comprising an N-glycan that has not been previously modified in vitro. In one aspect, the glycoprotein is an antibody.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The skilled artisan will understand that the drawings, described below, are for illustration purposes only. The drawings are not intended to limit the scope of the present teachings in any way

FIG. 1 shows the amino acid sequence of the full length α-L-fucosidase from Omnitrophica bacterium (449 aa). The first 22 amino acids comprise a signal sequence for protein secretion. The underlined portion of the protein (amino acids 23-359) contains the catalytic domain of the enzyme based on sequence alignments with other fucosidases (FIG. 2) and structural analysis (FIG. 3).

FIG. 2 shows a multiple alignment of the N-terminal part of mammalian FUCA1/2 enzymes (a.k.a. bovine kidney fucosidase (BKF)) and Omnitrophica α-L-fucosidase. Areas of significant difference between the prokaryotic Omnitrophica fucosidase and mammalian fucosidases are identified with boxes. These regions may correlate with each enzyme's different ability to react with labeled N-glycans in which the label is linked to the N-glycan via a glycosylamine linkage or other linkages. From top to bottom: SEQ ID NOs: 2-11, SEQ ID NO: 1.

FIG. 3 shows a tertiary structure models of bovine FUCA1 and Omnitrophica fucosidase created by SWISS-MODEL using a structure of Thermotogo maritima α-L-fucosidase as a template. For the Omnitrophica enzyme, the location of catalytic acid/base in the mobile loop (GLU264), as well as other differences indicated in the figure are believed to correlate with the differences in substrate recognition between these enzymes.

FIGS. 4A-4E show different types of labeling of N-glycan.

FIG. 4A shows how a label (2-AB) can be attached to the reducing end of an N-glycan via a Schiff base condensation reaction. This reaction has many steps, e.g., a hydrolysis reaction and a Schiff base condensation reaction, both of which are slow. As such, labeling N-glycans by Schiff base condensation typically takes longer than an hour.

FIG. 4B shows how a label (“InstantAB”) can be attached to the reducing end of an N-glycan via activated ester chemistry, where the N-hydroxysuccinimidyl is the leaving group. In this reaction, the amine in the N-glycan in its glycosylamine form attacks the ester carbonyl group to produce a carbamide linkage that links the label and the N-glycan, while the N-hydroxysuccinimidyl leaves.

FIG. 4C shows examples of alternative amine functionalized labels that can be added by Schiff base condensation. Each of these examples contains an amine (NH₂) to which a leaving group (e.g., a succinimide group) can be added, thereby producing an amine-reactive label that has an activated group that can undergo a nucleophilic substitution reaction with glycosylamine form of sugars. Examples include procainamide (Proc), APTS, 2-AA, AMAC and AP.

FIG. 4D provides a generic formula for some activated labels that can be used in the rapid labeling method. In this example, R is fluorescent label that may optionally contain a charge tag (for mass spectrometry analysis). In this example, the activated labels contain an N-hydroxy succinimidyl carbamate group, although other leaving groups could be used. Examples include AQC (fluorescent label) and Rapifluor™-MS (fluorescent label and charge tag) (Waters, Milford, Mass.), the structures for which are shown.

FIG. 4E shows the structures of several alternative amine-reactive labels (i.e., labels that have a reactive group attached to them, where the reactive group can react with an amine to form a covalent bond) that could be used herein.

FIG. 5 shows two alternative labeling methods of the enzymatically released N-glycans. The N-glycan can be cleaved from a glycoprotein using PNGase F. The released N-glycan can be subjected to low pH and linked to a fluorophore via a Schiff base condensation reaction and reduction (shown on the left) or via an amine reactive chemistry (shown on the right).

FIG. 6 shows the chromatographic profiles of UPLC-separated N-linked glycans of human IgG treated with different fucosidases. The N-glycans are labeled with RapiFluor-MS via a carbamide linkage. The arrows show that Omnitrophica fucosidase cleaves alpha 1,6 fucose from the N-glycans resulting in changes in their migration. Little or no effect was observed with BKF or with Fibrella fucosidase over the same time period.

FIG. 7 shows the chromatographic profiles of a UPLC-separated N-glycan substrate (NA2F-2AB) after treatment with equal amounts of different fucosidases over time. NA2F-2AB has been labeled with the traditional label 2-aminobenzamide via an amide bond. Omnitrophica fucosidase cleaves alpha 1,6 fucose from NA2F-2AB rapidly (within 1 hour) while commercial BKF (Prozyme, Hayward, Calif. or New England Biolabs, Ipswich, Mass.) does not achieve complete cleavage until between 3 and 16 hours.

FIGS. 8A-8C shows the chromatographic profiles of UPLC-separated N-glycan substrates (NA2F) having different labels, and treatment with different fucosidases. In each panel, NA2F has been labeled with a different compound via a carbamide linkage. Increasing concentrations of Omnitrophica fucosidase, Prozyme BKF and New England Biolabs BKF were compared over 16 hour incubations. The structures of the substrate and product N-glycans depicted on the top of the panels.

FIG. 8A shows results for NA2F-InstantAB plus fucosidase.

FIG. 8B shows results for NA2F-Instant Procainamide (InstantPC™) plus fucosidase.

FIG. 8C shows results for N-glycan-RapiFluorMS plus fucosidase.

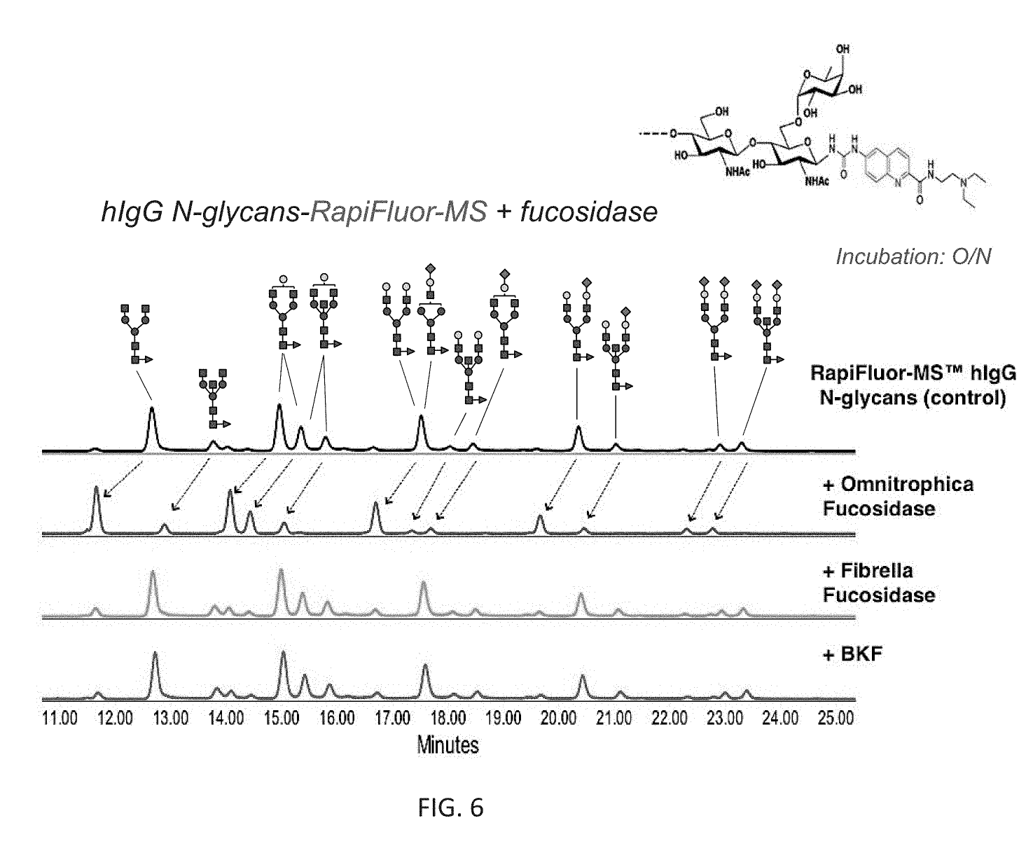

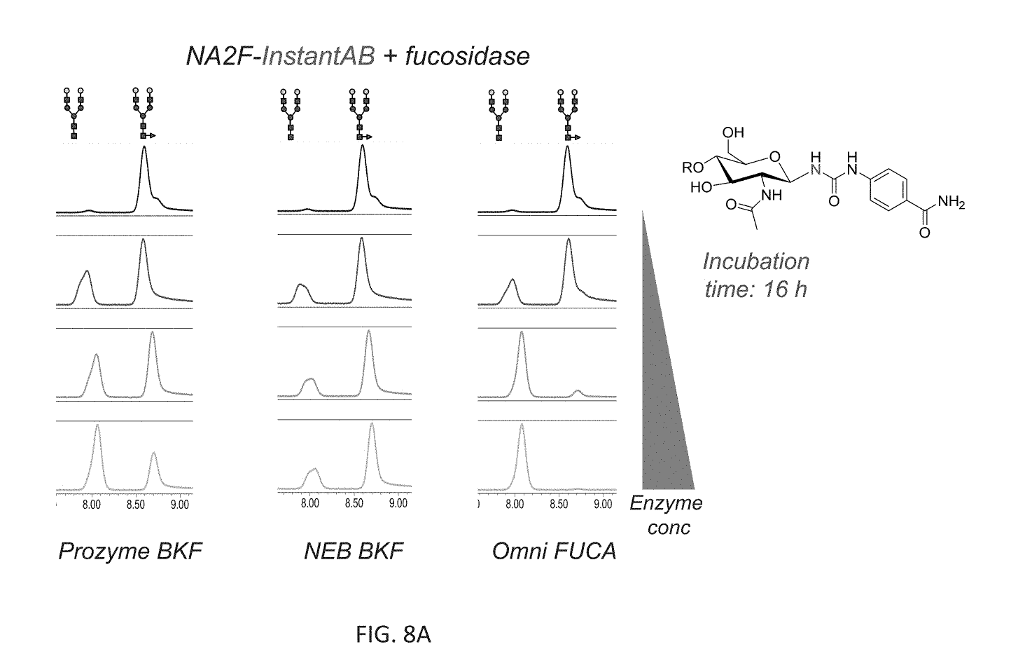

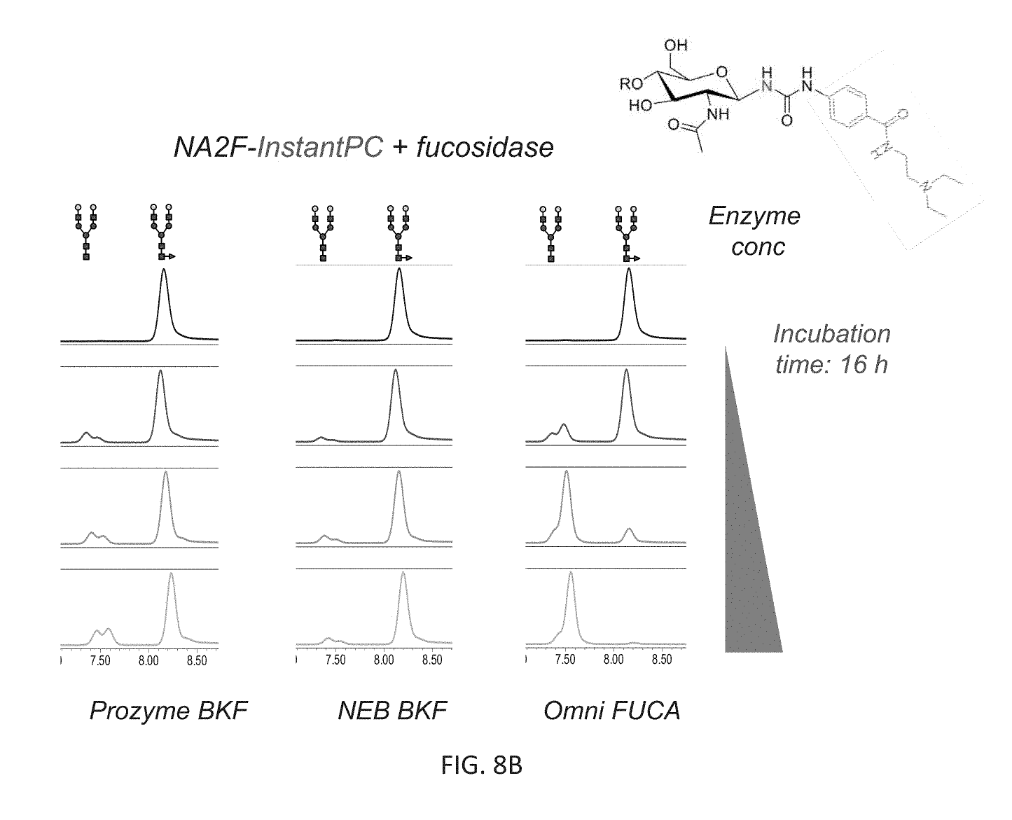

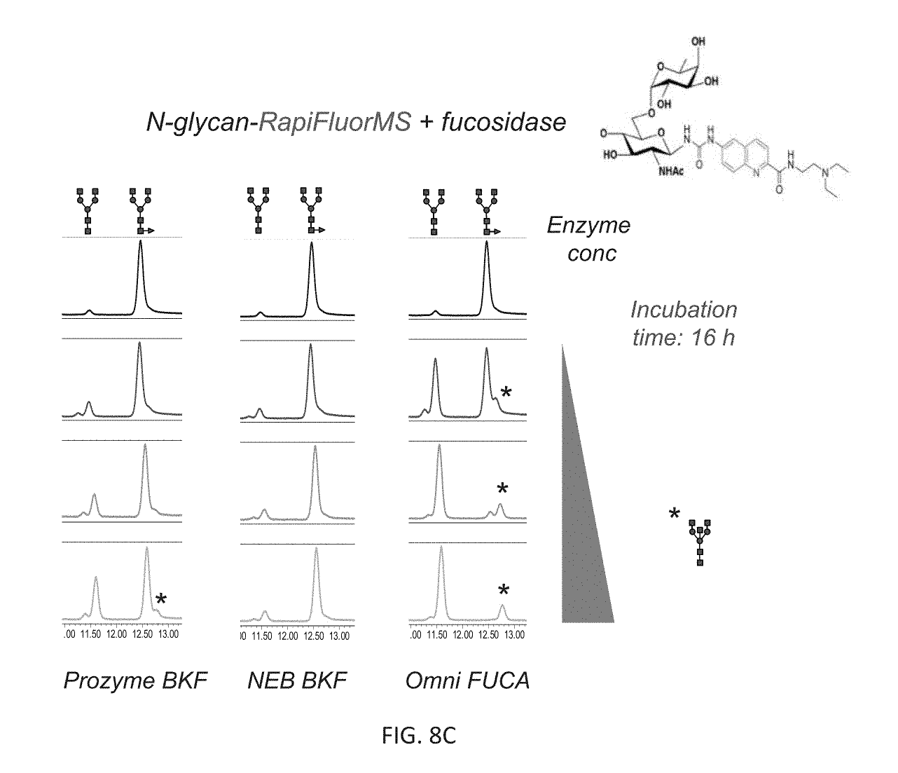

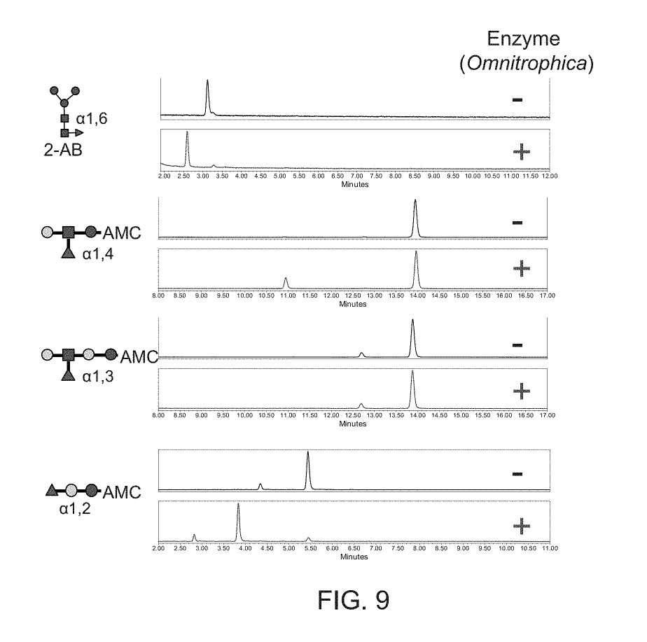

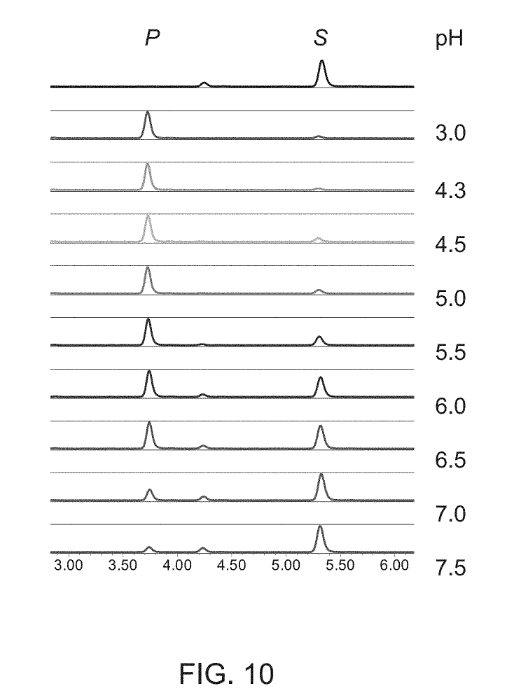

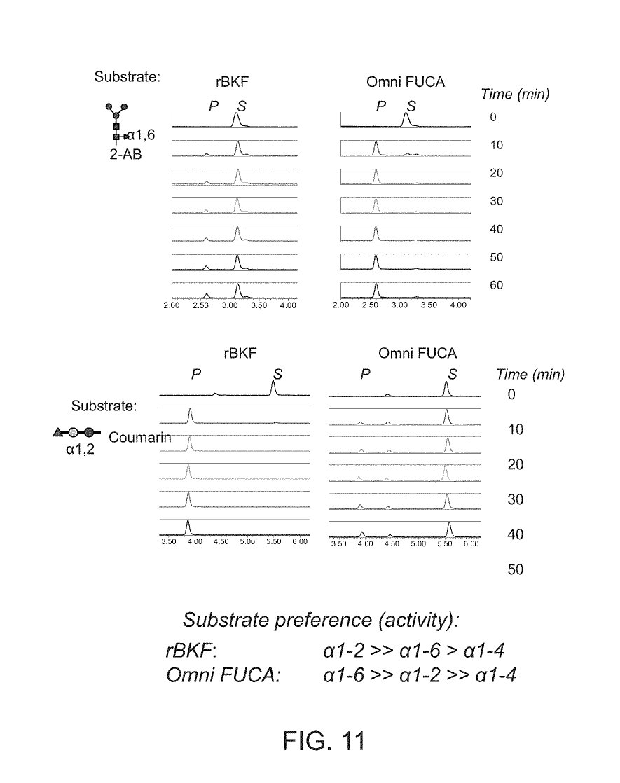

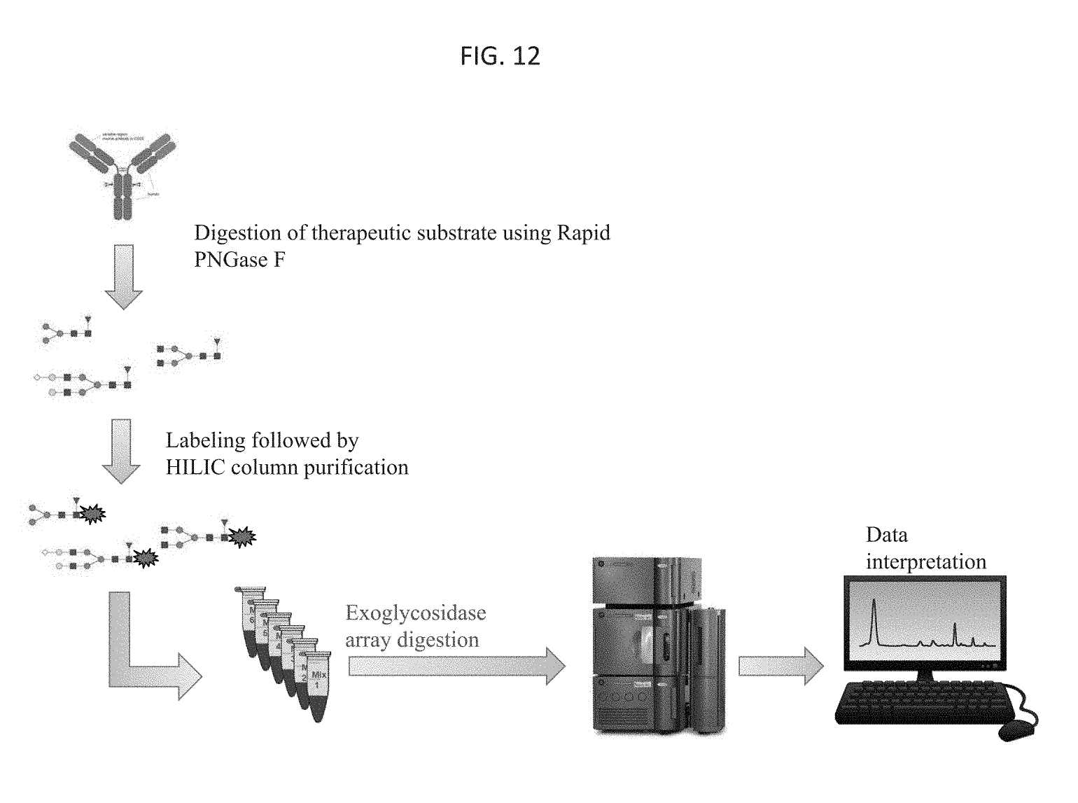

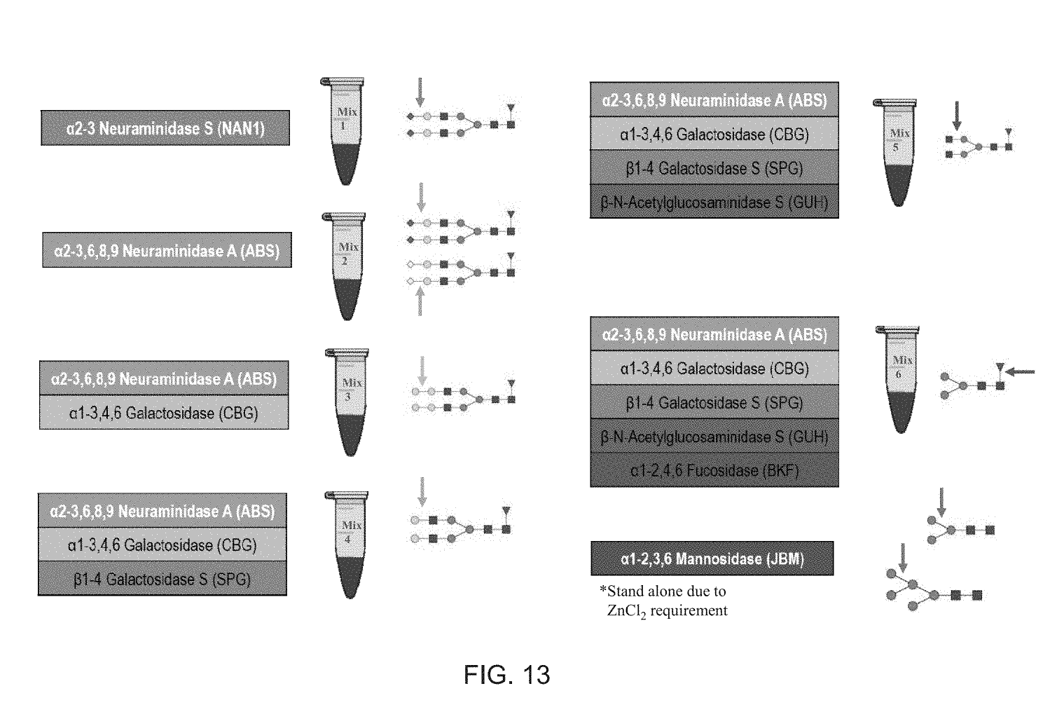

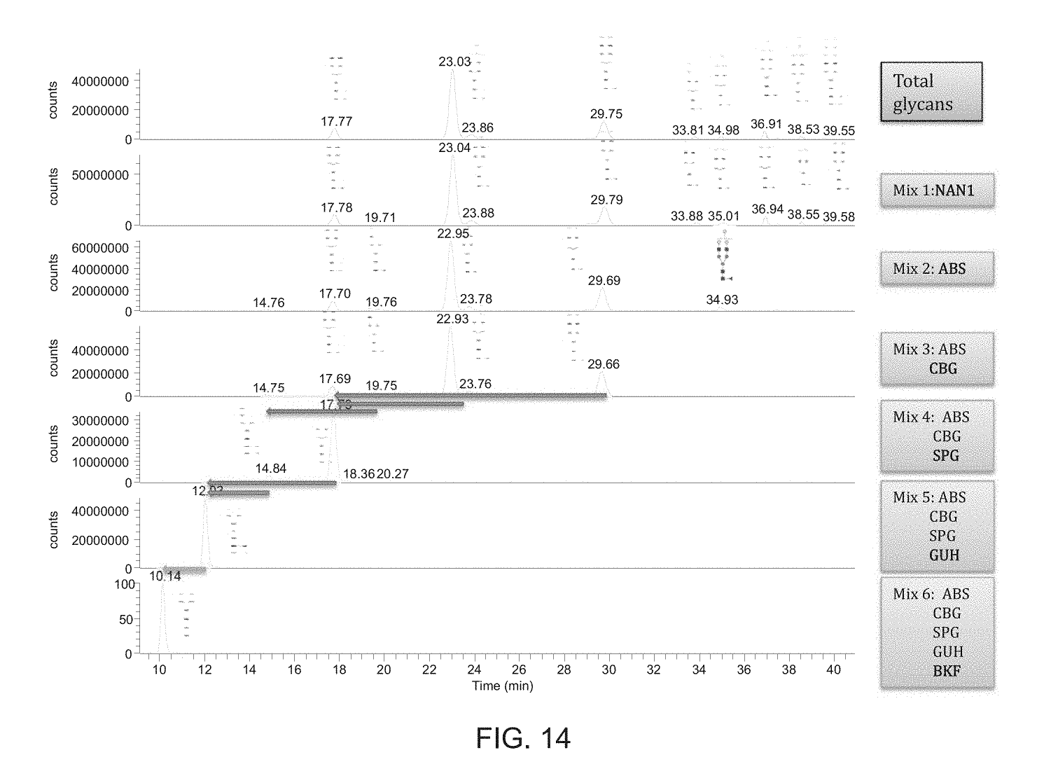

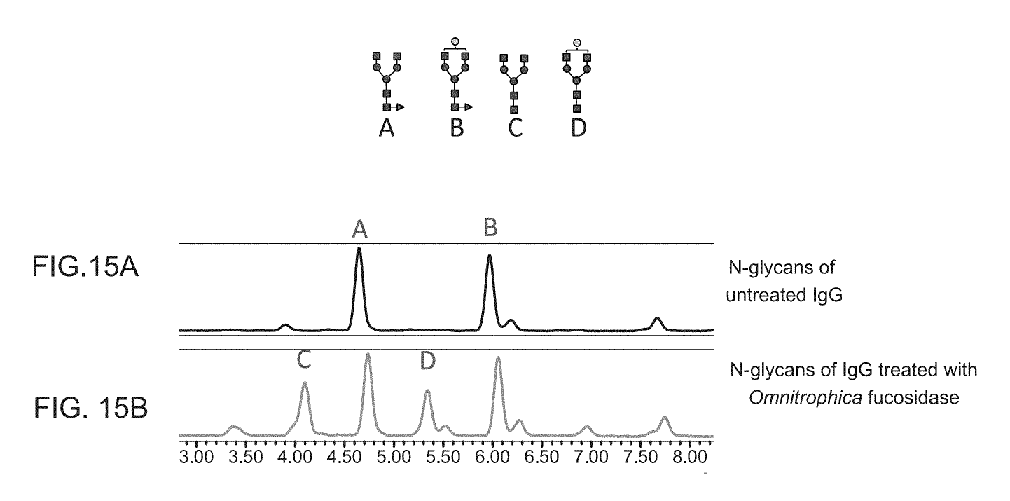

Only Omnitrophica fucosidase was able to effect complete cleavage of fucose from every NA2F conjugated to different dyes that contains a carbamide linkage. These results also show that the Omnitrophica fucosidase is significantly more active in cleaving alpha 1,6 fucose than the BKF fucosidase at the comparable concentrations. Note that in FIG. 8C, complete defucosylation of one N-glycan (NGA2F) is highlighted, although the reaction mix contained a plurality of N-glycans released from human IgG (as shown in FIG. 6). The additional peak in the chromatogram (depicted by asterisk) is due to removal of core fucose from a different N-glycan.

FIG. 9 shows the activity of purified Omnitrophica fucosidase on different coumarin-labeled oligosaccharides or 2-AB labeled (M3N2F) N-glycan substrates. The results show that Omnitrophica fucosidase is active on α1,6-, α1,2- and α1,4-linked fucose, but has no activity on α1,3-fucose.

FIG. 10 shows an activity assay using purified Omnitrophica fucosidase and 2′-Fucosyllactose-AMC glycan in buffers of varying pH values ranging from pH 7.5-pH 3.0. Peaks are labeled: S (substrate); and P (product). Optimal activity was observed at a pH below 5.5 and as low as pH 3.0.

FIG. 11 shows that the glycosidic bond preference of recombinant BKF and Omnitrophica fucosidases are different. Omnitrophica fucosidase cleaves the core a1,6-fucose specifically and very rapidly (within less than 10 minutes) from N-glycans which was labeled using conventional Schiff base labels such as 2AB. (Peaks are labeled: S (substrate); and P (product))

FIG. 12 shows an example of a conventional workflow for exo-array digestion of glycans from antibodies.

FIG. 13 shows the composition of an exemplary exoglycosidase array for sequencing antibody N-glycans.

FIG. 14 shows on example of how arrays of exoglycosidases (see FIG. 13) are used to sequence N-glycans (here using Schiff base labeled procainamide conjugated to N-glycans) where the N-glycans have been released from a mouse antibody.

FIG. 15A and FIG. 15B show release of the core fucose from the complex glycans covalently attached to a glycoprotein (murine IgG) where the complex glycans have not been previously modified by reagent enzymes in vitro.

FIG. 15A shows data from UPLC-HILIC-FLR with peaks A and B corresponding to the complex fucosylated glycans covalently attached to the anti-MBP monoclonal antibody (murine IgG2a) under non denaturing conditions.

FIG. 15B shows data from UPLC-HILIC-FLR with peaks C and D after treatment with Omnitrophica fucosidase under otherwise similar conditions as FIG. 15A where a1,6-linked core fucose has been liberated from the complex N-glycans covalently attached to glycoprotein.

### DESCRIPTION OF EMBODIMENTS

Unless defined otherwise herein, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

All patents and publications, including all sequences disclosed within such patents and publications, referred to herein are expressly incorporated by reference. Numeric ranges are inclusive of the numbers defining the range. Unless otherwise indicated, nucleic acids are written left to right in 5′ to 3′ orientation; amino acid sequences are written left to right in amino to carboxy orientation, respectively. The headings provided herein are not limitations of the various aspects or embodiments of the invention. Accordingly, the terms defined immediately below are more fully defined by reference to the specification as a whole. Unless described otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, New York. (1991) provide one of skill with the general meaning of many of the terms used herein. Still, certain terms are defined below for the sake of clarity and ease of reference.

In one embodiment, analysis of the N-glycan composition of a glycoprotein occurs after cleaving the N-glycan from the glycoprotein using an enzyme (e.g., PNGase F). The reducing end of the N-glycan (i.e., the end that was attached to the protein) may then be labeled for example, by linking a fluorescent label to produce a conjugate. Following column purification of the labeled conjugate, exoglycosidase digestion follows either using single enzymes or arrays. The conjugate may then be digested with one or more exoglycosidases that have cleavage specificity for terminal glycosidic bonds of specific sugars. The result of the exoglycosidase reaction is removal of the sugar from the non-reducing ends of the N-glycan. This changes the properties of the labeled conjugate. The released sugars and/or the remaining labeled N-glycan can be analyzed quantitatively by an analytical technique such as liquid chromatography (LC), capillary gel electrophoresis, mass spectrometry or LC-MS or the combination of LC-MS, CE-MS, etc. For example, this approach can be used to detect cleaved core α1,6 fucose and/or the cleaved N-glycan after treating with α-fucosidase.

Because each exoglycosidase has a defined specificity for a certain type of sugar, its stereochemical form (alpha or beta) and the specific site of its attachment to its adjacent sugar, exoglycosidase treatment yields structural information about the N-glycan. For example, treatment of an N-glycan that contains core α1,6 fucose with α-fucosidase causes removal of the fucose and a diagnostic shift in the N-glycan's chromatographic mobility that confirms the presence of this sugar.

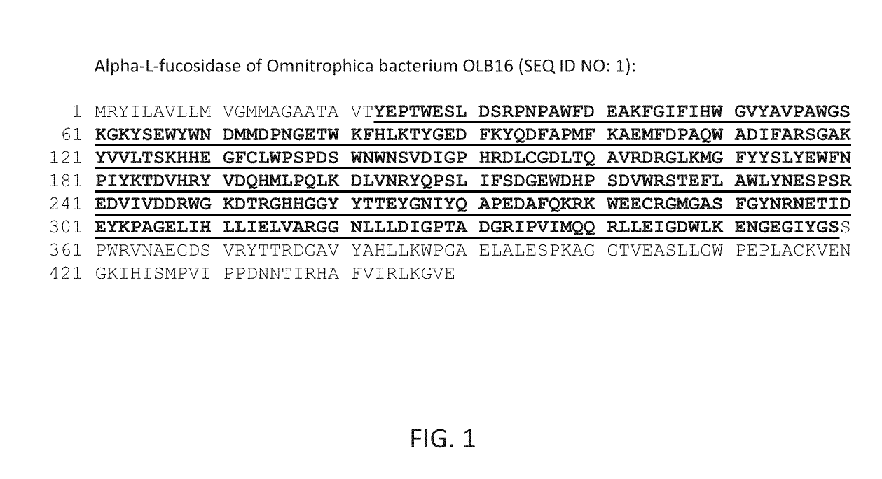

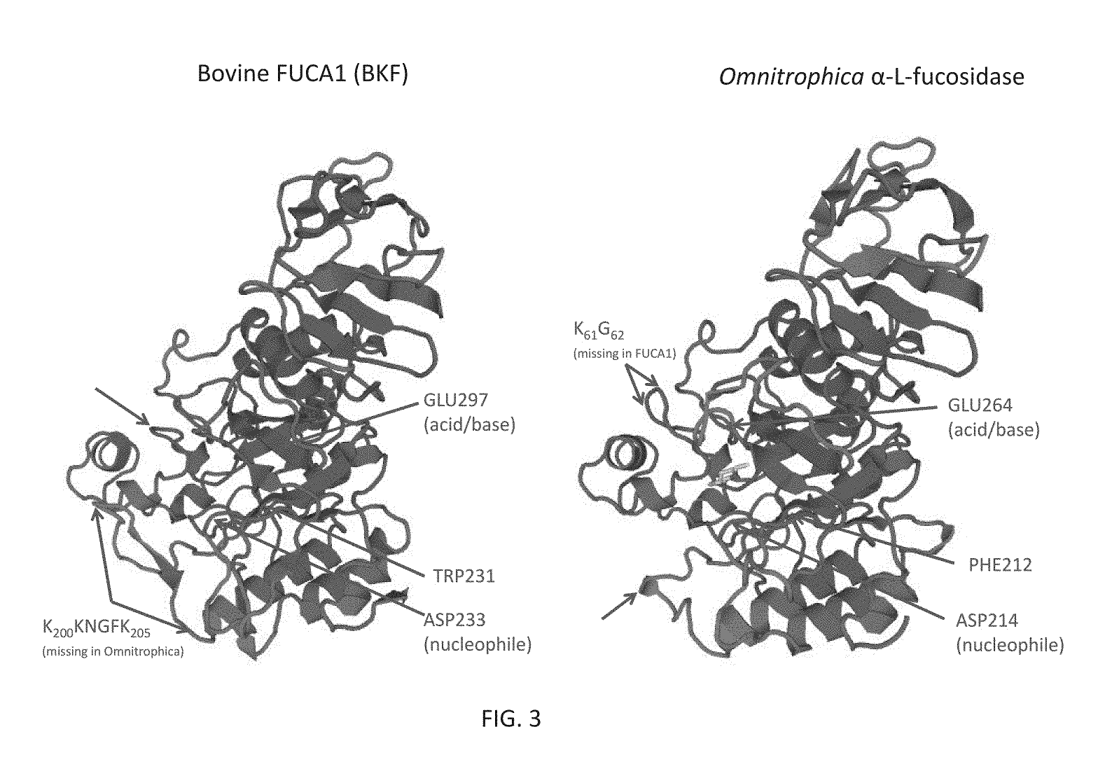

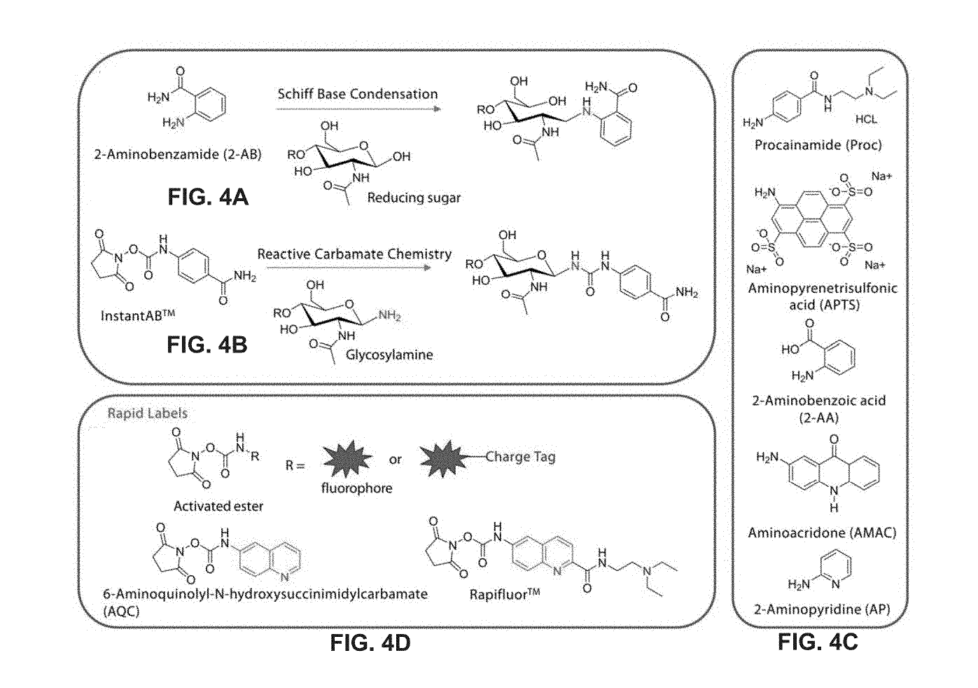

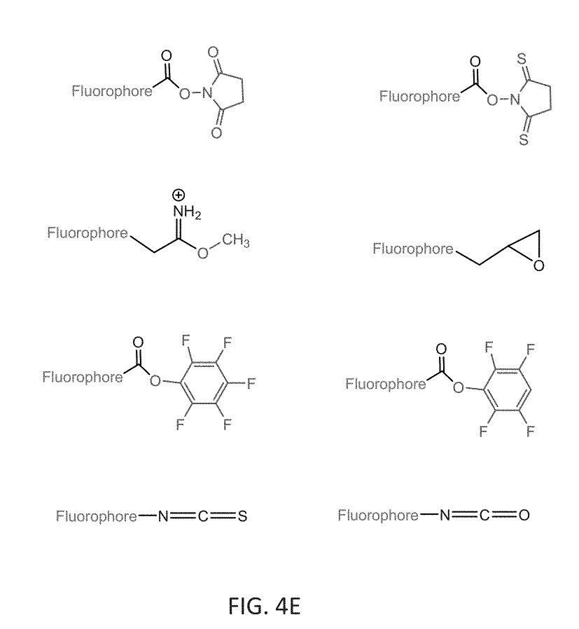

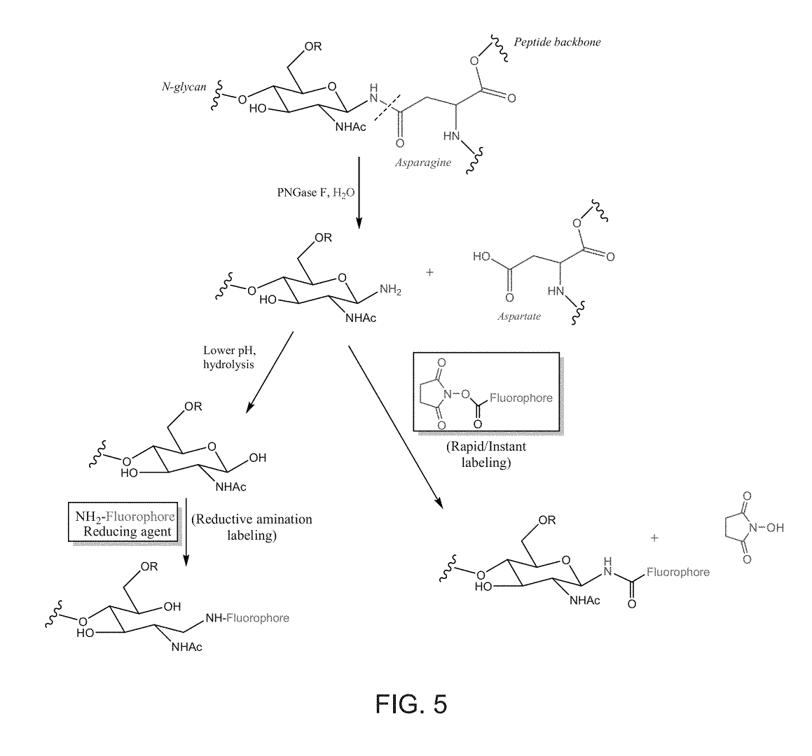

To exemplify various aspects of embodiments, FIGS. 1-3 show details of Omnitrophica fucosidase compared to other fucosidases. This includes sequence comparisons that highlight differences of note between the prokaryotic Omnitrophica fucosidase and mammalian fucosidases that may correlate with functional differences. Structural comparisons are provided in FIG. 3, which also show how the 3D structure may permit the Omnitrophica fucosidase to react with conjugates having carbamide bonds where BKF fucosidase is substantially inactive. FIGS. 4A-4E, FIG. 5 show details of the substrate chemistry that places a label or tag onto the reducing end of an N-glycan. Examples of the interaction of various fucosidases with various substrates under conditions specified in the description of figures and the examples are shown in FIGS. 6-11 and FIGS. 15A-15B. Standard sequencing workflows are shown in FIGS. 12-14.

As used herein, the term “label” refers to a non-naturally occurring detectable moiety that facilitates detection of the entity to which it is joined. For example, a label may be optically detectable and, as such, may contain a chromophore or a fluorophore. In other embodiments, the label may contain a mass tag or a charge tag (i.e., a tag of known charge that can be detected in mass spectrometry) or the like. In some embodiments, a label may contain a fluorophore and a charge tag.

Schiff base chemistry has been traditionally used to attach these labels to the reducing end of N-glycans. In this reaction, an amine functionalized label reacts in a condensation reaction with the aldehyde group of the glycan, resulting in a Schiff base, which can be reduced to yield amide bond. This labeling chemistry leaves the sugar ring open (FIGS. 4A-4E, FIG. 5). In addition to being a relatively slow reaction, other disadvantages include toxicity of reagents and incomplete reaction steps following Schiff base formation.

Recently, a new generation of labels has been developed that significantly improve the speed of glycan labeling and the sensitivity in certain detection methods (e.g. liquid chromatography and/or mass spectrometry). These labels include 6-aminoquinolyl-N-hydroxysuccinimidyl carbamate (AQC, Cohen, Analytical Biochemistry 1993: vol 211 pp279-287), RapiFluorMS (WO 2013049622), InstantAB (US 2012/0107942), InstantPC (US 2012/0107942) and Instant APTS (US 2012/0107942). These labels can be attached to the reducing end of an N-glycan using reactive carbamate chemistry to form a carbamide linkage with the N-glycan. This chemistry fluorescently labels the reducing end GlcNAc, but leaves its ring closed (FIGS. 4A-4E, FIG. 5).

Carbamate chemistry also referred to herein as activated ester chemistry or reactive carbamate chemistry refers to a chemical reaction in which an alkoxy or aryloxy group of an ester that contains a fluorophore serves as the leaving group, while the primary amine of the glycosylamine from the sugar serves as the nucleophile. The end result is the sugar is attached to the fluorophore through an amide bond or other bonds. Carbamate chemistry also encompasses reactions where the reactive group on the fluorophore is an epoxy, isothiocyanate, or isocyanate. While this chemistry has many advantages in N-glycan analysis, it has been found that BKF that is commonly used for N-glycan analysis does not cleave fucose or cleaves fucose very slowly from these labeled conjugates.

Examples of exoglycosidases that have specificity for certain monosaccharide linkages and anomericity (α/β) and are used in N-glycan analysis involving Schiff base labeled conjugates include α2-3 neuraminidase S, α2-3,6,8,9 neuraminidase A, α1-3,4,6 galactosidase, β1-4 galactosidase, P-N-acetylglucosaminidase S, α1-2,3,6 mannosidase; α-N-Acetylgalactosaminidase; α1-3,4 Fucosidase; β-N-Acetylhexosaminidase; β1-3 Galactosidase (New England Biolabs, Ipswich, Mass.) and other commercially available exoglycosidases. FIG. 13 shows an example of a panel of exoglycosidases, although other panels could be used. Common enzymes used for N-glycan sequencing include: Arthrobacter ureafaciens sialidase (ABS), almond meal alpha fucosidase (AM F), bovine testes beta galactosidase (BTG), coffee bean alpha galactosidase (CBG), Streptoccous pneumoniae beta hexosaminidase (SPH), jack bean alpha mannosidase (JBM), Streptoccous pneumoniae beta neuraminidase (NAN1), Streptoccous pneumoniae beta galactosidase (SPG), Helix pomatia beta-mannosidase, Aspergillus saitoi alpha-mannosidase and BKF that is preferably replaced by Omnitrophica fucosidase for the reasons give herein.

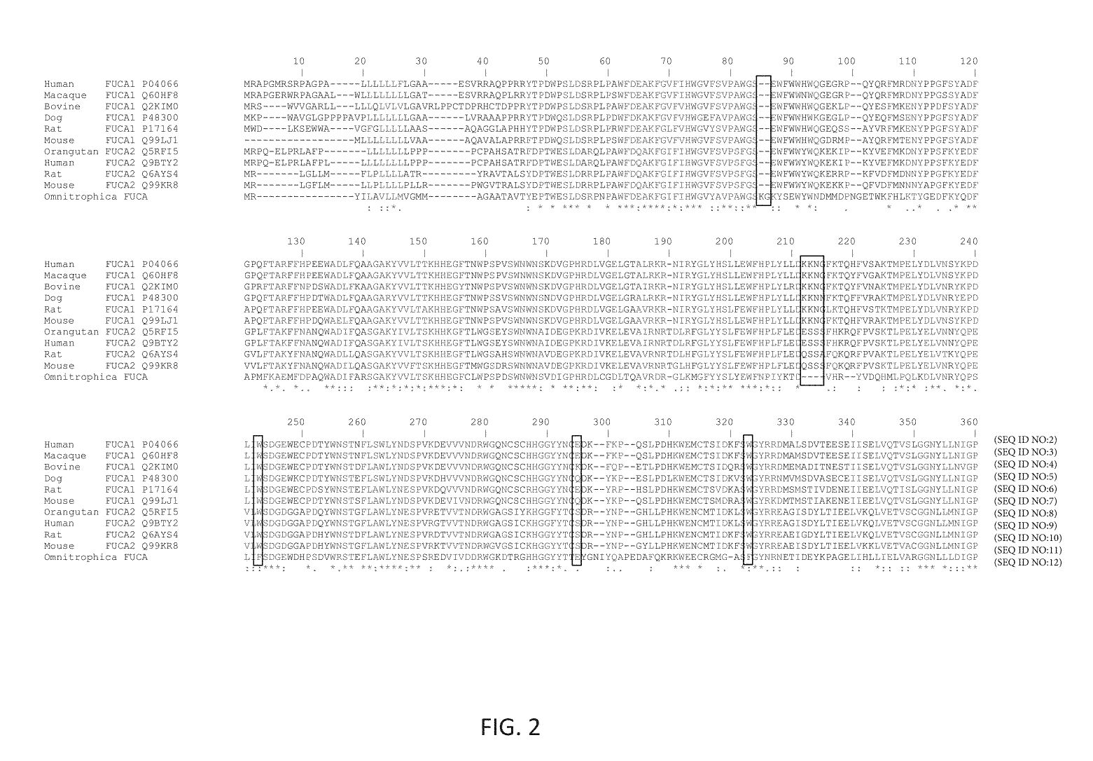

After substantial screening of fucosidases, a prokaryotic fucosidase was identified that was found to be more effective in cleaving fucose on complex N-glycans than previously preferred eukaryotic fucosidases. This fucosidase has an amino acid sequence that is at least 80% identical to (e.g., at least 90%, at least 95% or 100% identical to) amino acids 23-359 of sequence of SEQ ID NO:1 , the wild type Omnitrophica OLB16 α-fucosidase (as shown in FIG. 1). This part of SEQ ID NO:1 contains the catalytic domain based on sequence alignments with other fucosidases (FIG. 2) and structural analysis (FIG. 3) (also see EC 3.2.1.127 using IUBMB enzyme nomenclature). In some embodiments, the enzyme may have an amino acid sequence that is at least 80% identical to (e.g., at least 90%, at least 95% or 100% identical to) amino acids 23-449 of SEQ ID NO:1.

However as discussed further below, this glycosidase functionally differs in its catalytic properties from BKF and other eukaryotic fucosidases. This fucosidase has a preference for cleaving a α1,6 linkage over cleavage of other glycosidic linkages such as in α 1,2- and α1,4 between an α-L-fucose and an N-acetyl-D-glucosamine in an N-glycan (see for example, FIG. 11). The α-fucosidase of the bacterium Omnitrophica OLB16 rapidly cleaves core α1,6 linked fucose from N-glycans that have been labeled with new generation labels via a carbamide linkage. This enzyme has a pH optimum between pH 3 and pH 5 (see for example, FIG. 10). In one embodiment, the reaction mix containing a fucosidase and one or more other enzymes has pH 3.0-pH 5.5.

The Omnitrophica α-fucosidase and variants thereof may be used herein as a single reagent in a buffer, a component in an enzyme mixture or a component in a reaction mix containing a glycoprotein/peptide/N-glycan where one or more additional exoglycosidases may be additionally added. These additional exoglycosidases may be selected from any of exoglycosidases described above. A plurality of exoglycosidases can be used in serial digests of N-glycans or in arrays of enzyme mixtures (see for example, FIG. 13 showing standard exoglycosidase panels for sequencing of N-glycans from mouse antibody). In some embodiments, one or more neurominidases or exoglycosidases were expressed in a prokaryotic expression system or in eukaryotic expression systems. An advantage of recombinant exoglycosidases over naturally occurring purified exoglycosidases includes the absence of N-glycans on the recombinant enzymes that might confuse analysis of the target glycoprotein or peptide.

In general, exoglycosidase reactions can be performed on N-glycans that have been labeled using a Schiff base condensation reaction. These reactions take place under conditions that are compatible with enzyme activity. For example, pH, temperature, reaction solution components and concentration (e.g., salt, detergent, etc.), and length of reaction time can be optimized in order to achieve a desired level of exoglycosidase activity. In some embodiments, simultaneous digestion with multiple exoglycosidases can be used to analyze glycan structure and/or function. In some cases, simultaneous digestion can be performed in order to determine the presence of particular types of linkages and/or glycan modifications.

The Omnitrophica fucosidase and variants thereof has been found to have improved properties over the commercial BKF that does not cleave or inefficiently cleaves fucose on an N-glycan if the N-glycan is labeled with a dye using carbamate chemistry. While not wishing to be limited by theory, it is thought that the carbamate linkage produced by the reaction of the reactive carbamide of the label and the amine of the N-glycan results in a “closed ring” form of the labeled GlcNAc that impedes the bovine kidney fucosidase. In addition or alternatively, the molecular size, structure and/or charge of the label may be responsible for the observed differences in enzyme recognition of substrate and/or kinetics for different fucosidases. The identification of improved properties of Omintrophica fucosidase greatly facilitates the use of the new generation of labels for many analyses, including exoglycosidase-based glycan sequencing and other applications in which the new generation of labels are used. Glycans labeled with “instant” labels that utilize reactive carbamate chemistry may be incubated with fucosidase for as much as 18 hours. In one embodiment, the incubation may be less than 18 hours, for example less than 15 or 12 or 8 or 6 or 4 or 2 or 1 hour.

In addition, fucose cleavage from N-glycan labeled molecules that utilize Schiff base chemistry has been greatly improved using Omnitrophica fucosidase and variants thereof because of the improved kinetics of the reaction can be completed in minutes instead of many hours thus accelerating analysis (e.g. sequencing). Embodiments include combining an α-fucosidase with a conjugate comprising an N-glycan and a label, to make a reaction mix, and incubating the reaction mix so as to cleave any core fucose from the N-glycan. The reaction may be completed in less than a few minutes (e.g., less than 1 hour, 30 minutes, 10 minutes or 5 minutes) when the N-glycans are labeled by means of Schiff Base chemistry (e.g., 2-AB).

Another advantage of Omnitrophica fucosidase and variants thereof is the ability for the first time of removing a fucose (e.g., a core fucose is an α1,6-linked fucose residue that is attached to the N-acetyl glucosamine moiety that is linked to asparagine on the protein) from an N-glycan (core N-glycan or complex N-glycan) in otherwise intact glycoprotein or peptide without any prior total or partial deglycosylation steps (an intact protein) to produce an engineered molecule. In preferred embodiments, core alpha 1,6 fucose may be removed from intact or complex N-glycans on mammalian immunoglobulins (FIGS. 15A-15B), a capability that BKF and other fucosidases lack. The ability to engineer glycoproteins has been found to be important in the pharmaceutical industry where the composition of glycans on biotherapeutics can affect their performance or their rate of clearance from the bloodstream. For example, various studies have demonstrated that certain antibodies having N-glycans at Asn-297 that lack core α1,6 fucose are able to bind to the FcgRIIIa receptor with higher avidity than core fucosylated N-glycans. This stronger interaction enhances the antibody dependent cellular cytotoxicity (ADCC) response (Ferrara, C. et al. Biotechnol. Bioeng., 2006, 93, 851-861; Ferrara, C. et al. Proc. Natl. Acad. Sci. U. S. A., 2011, 108, 12669-12674). Existing enzymatic strategies first remove the bulk of an N-glycan by digestion with endoglycosidases such as endo S, endo F1, endo F2, endo H, or cocktails of these enzymes leaving a single N-acetylglucosamine residue to which core fucose is attached. This fucose can be accessed and removed by commercial fucosidases followed by glycoengineering back the other removed sugars (Collin, et al. EMBO J., 2001, 20, 3046-3055. Li, et al. J. Biol. Chem. 2016, 291, 16508-16518 and Tsai, et al. ACS Chem. Biol., 2017, 12, 63-72, WO2015/184008).

The term “complex N-glycan” as used here and in the claims refers to an N-glycan containing core sugars and also extra sugars. The term “intact” as used herein and in the claims refers to a naturally occurring N-glycan covalently linked to a protein or peptide. An intact N-glycan is intended to mean that it has not been previously enzymatically modified in vitro meaning it has not been cleaved or truncated by reagent enzymes in a reaction mix.

In certain embodiments, a kit may comprise: (a) the α1,6-fucosidase and (b) an amine-reactive label, as discussed above and shown by example in FIG. 4E. The various components of the kit may be present in separate containers or certain compatible components may be pre-combined into a single container, as desired.

In addition to the fucosidase, the kit may contain any of the additional components used in the method described above, e.g., a buffer, etc. In some embodiments, the kit may further comprise an enzyme for cleaving N-glycans off glycoproteins, e.g., PNGase F. In some embodiments, the kit may further comprise one or more exoglycosidases or mixtures of exoglycosidases in addition to the fucosidase, as listed above. In these embodiments, at least one of the one or more exoglycosidases may combined with the α1,6-specific fucosidase in an enzyme mixture, e.g., an enzyme mix described above. The amine-reactive label may contain an activated carbamate, although, as shown in FIGS. 4A-4E and FIG. 5, other chemistries may be used.

In addition to above-mentioned components, kits may further include instructions for using the components of the kit to practice the subject methods, i.e., instructions for sample analysis. The kit may also include components to isolate glycoproteins from culture media or biological samples (such as Protein A beads) and cartridges or plates that can purify labeled or unlabeled glycans.

Methods in accordance with the disclosure can be applied to glycans obtained from a wide variety of sources including, but not limited to, therapeutic formulations (e.g., antibodies erythropoietin, insulin, human growth hormone, etc.), commercial biological products (e.g., those presented in Table 5 of U.S. Pat. No. 8,729,241), and biological samples. A biological sample may undergo one or more standard analyses and/or purification steps prior to or after being analyzed according to the present disclosure.

In some embodiments, glycans from different batches of a therapeutic glycoprotein, whether prepared by the same method or by different methods, and whether prepared simultaneously or separately, are compared. In such embodiments, the present disclosure facilitates quality control of glycoprotein preparation.

In some embodiments, methods described herein can be used to characterize and/or control or compare the quality of therapeutic products. By way of example, the present methodologies can be used to assess glycosylation in cells producing a therapeutic glycoprotein. Particularly given that glycosylation can often affect the activity, bioavailability, or other characteristics of a therapeutic glycoprotein, methods for assessing cellular glycosylation during production of such a therapeutic glycoprotein are particularly desirable. Among other things, the present disclosure can facilitate real time analysis of glycosylation in production systems for therapeutic proteins.

Representative therapeutic glycoproteins whose production and/or quality can be monitored in accordance with the present disclosure include, for example, any of a variety of hematologic agents (erythropoietin, blood-clotting factors, etc.), interferons, colony stimulating factors, cytokines, antibodies, enzymes, and hormones. The term “glycoprotein” refers to any type of polypeptide that has been glycosylated, including peptides and protein complexes (e.g., antibodies, fusion proteins).

### EXAMPLES

Aspects of the present teachings can be further understood in light of the following examples, which should not be construed as limiting the scope of the present teachings in any way.

### Example 1

### Substrates and Methods for Glycan Labeling and Analysis

### I. Substrates

(a) Labeled N-glycans substrates used for specificity tests and activity assays were obtained commercially from Prozyme (Hayward, Calif.) and Waters (Milford, Mass.) as follows:

2AB-, InstantAB-, and InstantPC -labeled asialo-, galactosylated biantennary, core-substituted with fucose (NA2F-2AB, NA2F-InstantAB and NA2F-InstantPC, respectively); 2AB-labeled conserved trimannosyl core substituted with fucose (M3N2F-2AB) were from Prozyme; and human IgG N-glycans, RapiFluor-MS labeled were from Waters.

(b) Labeled oligosaccharides: Lacto-N-fucopentose III, 2′-Fucosyllactose (Prozyme, Hayward, Calif.) and Lacto-N-fucopentose II (Dextra Laboratories, Reading, UK) were labeled with 7-amino-4-methylcoumarin (AMC) as described below. AMC-labeled glycans were cleaned-up using gel filtration on G-25 column.

### II. Labeling of Selected Substrates

Schiff base condensation with 2-aminobenzamide (2-AB) (Sigma-Aldrich, St. Louis, Mo.) or 7-amino-4-methylcoumarin (AMC) (Sigma-Aldrich, St. Louis, Mo.) was used to label N-glycans and oligosaccharides as follows:

2-AB labeling mix for labeling N-glycans: 47.6 mg 2-AB in 300 μl glacial acetic acid was mixed with 62.8 mg NaCNBH4 in 700 μl, DMSO. (Final conc. of 350 mM 2-AB, 1 M sodium cyanoborohydride in 1 ml 7:3 DMSO:AcOH). 10 μl of labeling mix was added per 1 nmol of dried glycans and heated at 65° C. for 2 hours to result in a labeled N-glycan.

7-amino-4-methylcoumarin (AMC) labeling mix for labeling oligosaccharides: 20 mg AMC, 41 μglacial acetic acid, 35 mg sodium cyanoborohydride (NaCNBH4) and 300 μl methanol. 10 μl of the labeling mix was added per 3 nmol of dried N-glycans. This was heated at 80° C. for 45 minutes to label the N-glycan.

### III. Clean-Up of the Labeled N-Glycans Using SPE-HILIC Nest Cartridges

(MicroSpin™ columns, 10-100 μg capacity (The Nest Group, Southborough, Mass.))

Cartridges were equilibrated using 350 μl: 1× with acetonitrile (ACN), 2× water, 3×90% ACN/NH₄F, 2 minute spins at 110×g (800 rpm). Labeling samples (10 μl) were diluted up to 300 μl with 90% ACN/10% NH₄F, loaded on cartridge, and washed with 5×350 μl 90% ACN/NH₄F. Samples were eluted in 100 μl 50 mM NH₄F, pH 4.4.

IV. Analysis of N-Glycan Labeled Conjutes by Ultra-Performance Hydrophilic Interaction Liquid Chromatography with Fluorescence Detection (UPLC-HILIC-FLR)

Gradients and detection parameters vary according to the labeled substrate. In general, 2-AB labeled N-glycans or IntactAB-labeled N-glycans or IntactPC-labeled N-glycans (obtained according to Example 1 |(a)) or as a product of enzyme cleavage of a glycoprotein) or products of exoglycosidase digestion were separated by UPLC using a Waters Acquity BEH glycan amide column (2.1×150 mm, 1.7 μm) on a Waters H-Class ACQUITY instrument (Waters Milford, Mass.) equipped with a quaternary solvent manager and a fluorescence detector. Solvent A was 50 mM ammonium formate buffer pH 4.4 and solvent B was acetonitrile. The gradient used was 0-1.47 min, 30% solvent A; 1.47-24.81 min, 30-47% solvent A; 25.5-26.25 min, 70% solvent A; 26.55-32 min, 30% solvent A. The flow rate was 0.561 mL/min. The injection volume was 10 μL and the sample was prepared in 70% (v/v) acetonitrile. Samples were kept at 5° C. prior to injection and the separation temperature was 40° C. The fluorescence detection wavelengths were: λex=330 nm and λem=420 nm for 2-AB; λex=278 nm and λem=344 nm for InstantAB, λex=285 nm and λem =345 nm for InstantPC. Data collection rate was 20 Hz. All data was processed using Waters Empower 3 chromatography workstation software.

In general for RapiFluor-MS-labeled N-glycans, separation was carried out by UPLC using a Waters Acquity BEH glycan amide column (2.1×150 mm, 1.7 μm) on a Waters H-Class ACQUITY instrument equipped with a quaternary solvent manager and a fluorescence detector. Solvent A was 50 mM ammonium formate buffer pH 4.4 and solvent B was acetonitrile. The gradient used was 0-35 min, 25-46% solvent A; 36.5-39.5 min, 100% solvent A; 43.1-55 min, 25% solvent A. The flow rate was 0.4 mL/min. The injection volume was 5 μL and the sample was prepared in 75% (v/v) acetonitrile. The fluorescence detection wavelengths were λex=265 nm and λem=425 nm with a data collection rate of 20 Hz. All data was processed using Waters Empower 3 chromatography workstation software.

### Example 2

### Cloning, Expression and Properties of Omnitrophica α-L-Fucosidase

The DNA (GenBank: LMZT01000142.1, REGION: 29928..31277) encoding Omnitrophica α-L-fucosidase (Gen Bank: KXK31601.1) was synthesized in vitro and DNA fragment encoding 23-449 amino acids was cloned into bacterial expression vector pJS119K using NEBuilder® HiFi DNA Assembly Cloning Kit (New England Biolabs, Ipswich, Mass.). The assembled plasmid was used for the transformation of E. coli expression strain (NEB® Express Competent E. coli (New England Biolabs, Ipswich, Mass.)). Bacterial α-L-fucosidase was expressed by induction with 0.4 mM IPTG at 30° C. for 4 hours. The expressed protein purified using conventional liquid chromatography methods. A synthetic substrate 2-Chloro-4-nitrophenyl a-L-fucopyranoside (CNP-Fuc) was used to assay enzymatic activity during chromatography purification. 1 μl of α-L-fucosidase sample was added to 100 μl of 2 mM CNP-Fuc in 20 mM sodium acetate buffer, pH 5.5, and incubated 1 hour at 37° C. Absorbance readings were taken at 405 nm.

### Substrate Specificity and Substrate Preference of the Purified Omnitrophica Fucosidase

Substrate specificity of purified Omnitrophica fucosidase was determined using N-glycan or oligo saccharide substrates containing α1,6-linked fucose (M3N2F-2AB), α1,2-linked fucose (2′-Fucosyllactose-AMC), α1,3-linked fucose (Lacto-N-fucopentose III-AMC) or α1,4-linked fucose(Lacto-N-fucopentose II-AMC) (see Example 1). 2 pmol of 2AB-labeled glycan or 30 pmol AMC-labeled glycan was incubated with 0.5 μg of Omnitrophica fucosidase overnight at 37° C. in 50 mM sodium acetate, pH 5.5. At the end of incubation time, 100 μl of 20% acetonitrile was added to the mix to stop the reaction. The reaction mixture was transferred to the Nanosep 10K Omega centrifugal device and centrifuged for 5 minutes at 12,000×g. For UPLC-HILIC-FLR analysis, 5 μl of each sample was mixed with 11.7 μl acetonitrile (final ratio 30:70 water/acetonitrile). A 5-10 μl aliquot of this mix was used for UPLC-HILIC-FLR separation as described in Example 1(IV). The results (shown in FIG. 9) indicate that Omnitrophica fucosidase is active on α1,6-, α1,2- and α1,4-linked fucose, but shows no activity on α1,3-fucose.

The glycosidic bond preference of recombinant BKF (rBKF) (New England Biolabs, Ipswich, Mass.) and Omnitrophica enzymes were compared (see for example, FIG. 11). 20 pmol of 2AB-labeled glycan or 300 pmol of AMC-labeled glycan was incubated with 1 μg of Omnitrophica α-L-fucosidase or 1 μg of BKF in 50 mM sodium acetate buffer, pH 5.5 in a total reaction volume of 100 μl at 37° C. At the end of each time point, 10 μl aliquot was taken and the reaction was stopped by adding 100 μl of 20% acetonitrile. The reaction mixture was transferred to the Nanosep 10K Omega centrifugal device and centrifuged for 5 minutes at 12,000×g. For UPLC-HILIC-FLR analysis, 5 μL of each sample was mixed with 11.7 μl acetonitrile (final ratio 30:70 water/acetonitrile). A 5 μL aliquot of this mix was used for UPLC-HILIC-FLR separation as described in Example 1(IV). Activity assays with results described in FIG. 9 and FIG. 11 were performed in the 50 mM sodium acetate buffer, pH 5.5

Omnitrophica fucosidase exhibits highest activity on α1,6-linked fucose, followed by α1,2- and α1,4-fucose, whereas rBKF cleaves more efficiently α1,2-linked fucose than α1,6- and α1,4-fucose. This data also shows that under the conditions used the Omnitrophica fucosidase cleaves the core α1,6-fucose from 2AB-labeled N-glycans very rapidly (within 1 hour).

### Optimal pH of Omnitrophica Fucosidase

An activity assay was performed using 0.5 μg of purified Omnitrophica fucosidase and 30 pmol of 2′-Fucosyllactose-AMC substrate in buffers of varying pH values ranging from pH 7.5-pH 3.0. Following buffers were used: 50 mM glycine buffer, pH 3.0; 50 mM sodium acetate, pH 4.3; 50 mM sodium citrate, pH 4.5; 50 mM sodium acetate, pH 5.0; 50 mM MES, pH 6.0; 50 mM MES, pH 6.5; 50 mM sodium phosphate, pH 7.0; 50 mM sodium phosphate, pH 7.5. The reactions were incubated at 37° C. overnight and the samples were cleaned and analyzed by UPLC-HILIC-FLR as described in Example 1(IV). The results are shown in FIG. 10. Optimal activity was observed at a pH below 5.5 and as low as 3.0.

### Example 3

### Activity Assays

Fucosidase Activity Using Glycans Labeled with Fluorophore Dyes
I. Fucosidase Activity on an N-glycans Having a α1,6-Core Fucose Labeled with RapiFluor-MS.

Several α-L-fucosidases were tested for their ability to cleave off core fucose from the N-glycans labeled with one of the “instant” labels, RapiFluor-MS (see Example 1). Labeled N-glycans (20 pmol) were incubated overnight at 37° C. in the 50 mM sodium acetate buffer, pH 5.5 with different α-L-fucosidases (1 μg of each Omnitrophica fucosidase, FibreIla fucosidase and rBKF used).

After the fucosidase reaction, N-glycans were analyzed by UPLC-HILIC-FLR (see Example 1(IV)). The results are shown in FIG. 6. Removal of the core α1,6-fucose from N-glycan causes a diagnostic shift in the N-glycan's chromatographic mobility (indicated by arrows). The results indicate that only Omnitrophica fucosidase was able to remove completely core α1,6-fucose from the N-glycans labeled with RapiFluor-MS. Very little to no effect was observed with rBKF or with FibreIla fucosidase over the same time period. Also important to note that Omnitrophica fucosidase performed equally well on various types of core fucosylated, RapiFluor-MS-labeled N-glycans (bi-antennary, containing bi-secting GlcNAc, sialilated, etc.) present in the analyzed sample.

II. Fucosidase Activity on an N-Glycans Having a α1,6-Core Fucose Labeled with 2-Aminobenzamide (2AB)

α-L-fucosidases were compared for their ability to release core fucose from the N-glycans labeled with 2AB via reductive amination. 8 pmol of NA2F-AB was incubated at 37° C. in 40 μl of the 50 mM sodium acetate buffer, pH 5.5 with 0.11 μg of each native BKF, rBKF and Omnitrophica fucosidase. At the end of each time point (0, 1, 3, 16 hours), 10 μl aliquot was taken and the reaction was stopped by adding 100 μl of 20% acetonitrile.

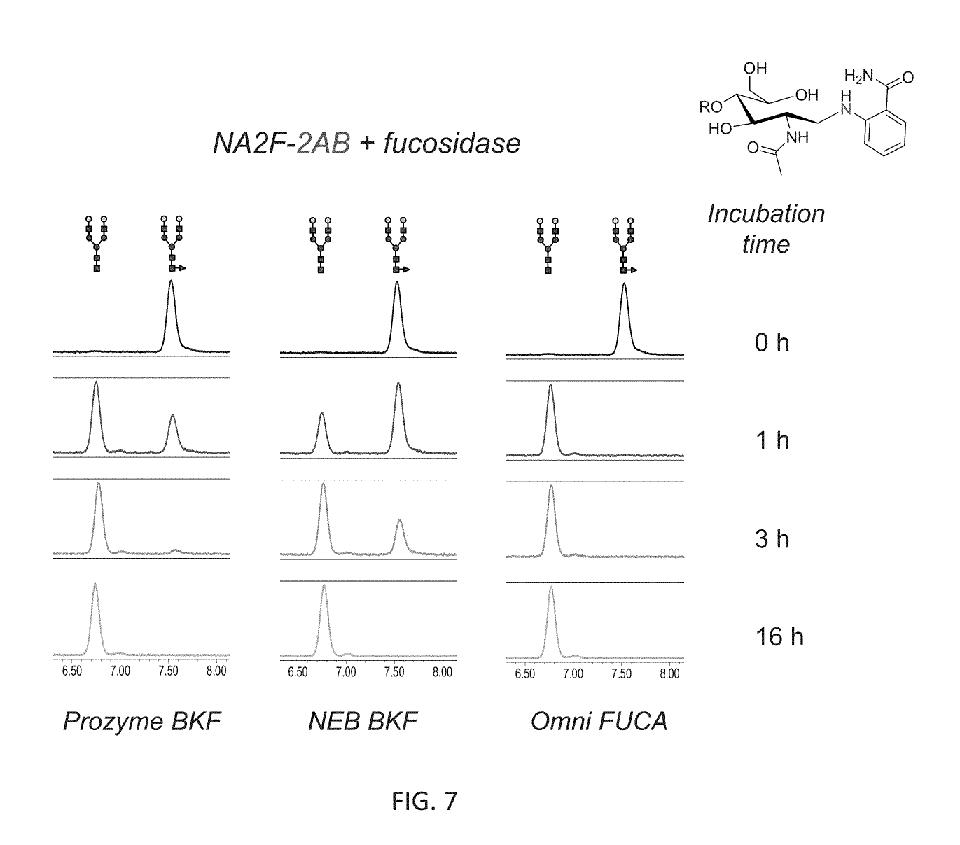

After the fucosidase reaction, N-glycans were analyzed by UPLC-HILIC-FLR as described in Example 1(IV). The results are shown in FIG. 7. Removal of the core α1,6-fucose from N-glycan causes a diagnostic shift in the N-glycan's chromatographic mobility. The data indicate the Omnitrophica fucosidase rapidly cleaves the core fucose from 2AB-labeled N-glycan within 1 hour while equal amount of the commercial fucosidases do not achieve complete cleavage until more than 3 hours. Thus, under the conditions tested Omnitrophica fucosidase demonstrates superior performance (speed and complete removal of core fucose) on the substrates with 2AB label.

III. Fucosidase Activity on N-Glycans Having a α1,6-Core Fucose Labeled with InstantAB, InstantPC, RapiFluor-MS Labels

The release of core fucose from N-glycans labeled with different “instant” labels was tested. 2 pmol of NA2F-InstantAB or NA2F-InstantPC, or 24 pmol of human IgG N-glycans labeled with RapiFluor-MS was incubated at 37° C. for 16 hours in 40 μl of the 50 mM sodium acetate buffer, pH 5.5 with increasing concentrations of each enzyme: 0, 6, 16, 30 mU of native BKF; 0, 20, 64, 120 U of rBKF and 0, 0.11, 0.8 and 2.2 μg of Omnitrophica fucosidase. At the end of incubation time, the reaction was stopped by adding 100 μl of 20% acetonitrile. The reaction mixture was cleaned-up using Nanosep 10K Omega centrifugal device and N-glycans were analyzed by UPLC-HILIC-FLR as described in Example 1(IV). The results are shown in FIGS. 8A-8C ((FIG. 8A) Instant 2-aminobenzamide (InstantAB); (FIG. 8B) InstantPC; (FIG. 8C) RapiFluor-MS).

The results show that, under the conditions used, efficient and complete removal of the core fucose from N-glycan with tested “instant” labels was attained only with Omnitrophica fucosidase while the commercial fucosidases do not achieve complete cleavage even at the highest concentrations tested. Again, this demonstrates a superior performance of Omnitrophica fucosidase on the substrates labeled with various “instant” labels (containing only fluorophore or fluorophore+charge tag).

### Example 4

### Enzymatic Release of α1,6-Linked Core Fucose from Complex N-Glycans Attached to Glycoproteins

Anti-MBP monoclonal antibody (murine IgG2a) was incubated with Omnitrophica fucosidase to investigate if this fucosidase could liberate α1,6-linked core fucose from glycoprotein under non-denaturing conditions.

After fucosidase treatment, N-glycans were released from protein using PNGase F, labeled with 2-AB and analyzed by UPLC-HILIC-FLR. The results presented in FIGS. 15A-15B show that Omnitrophica fucosidase can liberate α1,6-linked core fucose from the complex N-glycans which are covalently attached to glycoprotein at asparagine residues by an N-glycosidic bond.

More specifically, 15 μg of native murine anti-MBP antibody (New England Biolabs, Ipswich, Mass.) were mixed with 6.5 μg of α-L-fucosidase in 50 mM sodium acetate buffer, pH 6.0 in a 20 μl final reaction volume, and the reaction mixes were incubated at 37° C. for 36 hours. After the treatment, the reactions were stopped, diluted with 90 μl of water and the buffer exchanged to 50 mM sodium phosphate, pH 7.5 using Nanosep 10K Omega centrifugal devices (Pall Life Sciences, Port Washington, N.Y.). 2.5 μl of PNGase F was added to the filters and incubated at 37° C. for 1.5 hours. The released glycans were collected by centrifugation, dried, labeled with 2AB and cleaned-up as described in Example 1(II-III). The purified labeled glycans were further analyzed by UPLC-HILIC-FLR as described in Example 1(IV). Using this approach, different core fucosylated glycoproteins can be remodeled using Omnitrophica fucosidase.

It will also be recognized by those skilled in the art that, while the invention has been described above in terms of preferred embodiments, it is not limited thereto. Various features and aspects of the above described invention may be used individually or jointly. Further, although the invention has been described in the context of its implementation in a particular environment, and for particular applications those skilled in the art will recognize that its usefulness is not limited thereto and that the present invention can be beneficially utilized in any number of environments and implementations where it is desirable to examine analytes. Accordingly, the claims set forth below should be construed in view of the full breadth and spirit of the invention as disclosed herein.

What is claimed is:

1. A reaction mix comprising:
(a) an α-L-fucosidase having an amino acid sequence that is at least 90% identical to amino acids 23-359 of sequence of SEQ ID NO:1 ; and
(b) a conjugate comprising an N-glycan and a label.

2. The reaction mix of claim 1, wherein the label comprises a fluorophore and/or a charge tag.

3. The reaction mix of claim 1, wherein the N-glycan of the conjugate comprises a core fucose.

4. The reaction mix of claim 1, wherein the N-glycan is linked to the label via the reducing end of the N-glycan.

5. The reaction mix of claim 1, wherein the N-glycan is linked to the label via amine reactive chemistry or by a Schiff base condensation reaction.

6. The reaction mix of claim 1, wherein the N-glycan is from a therapeutic glycoprotein.

7. The reaction mix of claim 1, wherein the reaction mix further comprises:
(c) one or more exoglycosidases selected from the group consisting of:
α2-3 neuraminidase S,
α2-3,6,8,9 neuraminidase A,
α1-3,4,6 galactosidase,
β1-4 galactosidase,
β-N-acetylglucosaminidase S, and
α1-2,3,6 mannosidase.

8. The reaction mix of claim 1, wherein the reaction mix has a pH in the range of pH 3.0 to pH 5.5.

9. An enzyme mix comprising:
(a) an α-fucosidase having an amino acid sequence that is at least 90% identical to amino acids 23-359 of sequence of SEQ ID NO:1; and
(b) one or more exoglycosidases that are not obtainable from Omnitrophica selected from the group consisting of:
α2-3 neuraminidase S,
α2-3,6,8,9 neuraminidase A,
α1-3,4,6 galactosidase,
β1-4 galactosidase,
β-N-acetylglucosaminidase S, and
α1-2,3,6 mannosidase.

10. An enzyme mix according to claim 9, wherein none of the one or more exoglycosidases in the enzyme mix is glycosylated.

11. The enzyme mix of claim 9, wherein the enzyme mix comprises two or more of the exoglycosidases.

12. The enzyme mix of claim 9, wherein the enzyme mix comprises three or more of the exoglycosidases.

13. The enzyme mix of claim 9, wherein the enzyme mix comprises α2-3 neuraminidase S, α2-3,6,8,9 neuraminidase A, α1-3,4,6 galactosidase, β1-4 galactosidase and β-N-acetylglucosaminidase S.

14. A kit, comprising:
(a) an α-fucosidase having an amino acid sequence that is at least 90% identical to amino acids 23-269 of sequence of SEQ ID NO:1; and
(b) one or more exoglycosidases selected from the group consisting of:
α2-3 neuraminidase S,
α2-3,6,8,9 neuraminidase A,
α1-3,4,6 galactosidase,
β1-4 galactosidase,
β-N-acetylglucosaminidase S, and
α1-2,3,6 mannosidase.

15. The kit of claim 14, wherein the kit further comprises PNGase F.

16. The kit of claim 14, wherein at least one of the one or more exoglycosidases is combined with the α1,6-fucosidase in a reaction mixture.

17. A method for cleaving fucose from an N-glycan, comprising:
(a) combining:
(i) an α-fucosidase having an amino acid sequence that is at least 90% identical to amino acids 23-359 of sequence of SEQ ID NO:1 with
(ii) a conjugate comprising an N-glycan and a label to make a reaction mix; and
(b) incubating the reaction mix so as to cleave core α1,6-fucose from the N-glycan.

18. A method according to claim 17 wherein cleavage of the core fucose is substantially complete.

19. The method of claim 17, wherein step (a) is done at a pH in the range of pH 3.0 to pH 5.5.

20. The method of claim 17, wherein the method further comprises: (c) detecting the cleaved glycan or the cleaved fucose after step (b).

21. The method of claim 17, wherein the detecting is quantitative.

22. The method of claim 17, wherein the detecting is done by liquid chromatography, mass spectrometry, capillary electrophoresis or any combination thereof.

23. The method of claim 17, wherein the method further comprises:
cleaving a N-glycan from a glycoprotein; and
conjugating the reducing end of the N-glycan with an amine-reactive label or an amine functionalized label to produce a labeled conjugate.

24. The method of claim 17, wherein the cleaving of the N-glycan from the glycoprotein is done using PNGase F.

25. The method of claim 17, wherein the protein is a therapeutic glycoprotein.

26. A method for cleaving fucose from an N-glycan, comprising:
(a) combining an α-fucosidase having an amino acid sequence that is at least 90% identical to amino acids 23-359 of sequence of SEQ ID NO:1 with an intact N-glycan linked glycoprotein or glycopeptide in a reaction mix; and
(b) incubating the reaction mix so as to cleave any core α1,6-fucose from the N-glycan.

27. A method according to claim 26, wherein the glycoprotein is an antibody.

28. A reaction mix comprising:
(a) an α-L-fucosidase having an amino acid sequence that is at least 90% identical to amino acids 23-359 of sequence of SEQ ID NO:1; and
(b) a glycoprotein or glycopeptide comprising an N-glycan that has not been previously modified in vitro.

29. A reaction mix according to claim 28, wherein the glycoprotein is an antibody


SEQUENCE LISTING (12 sequences)

SEQ ID NO: 1 - protein, 449 aa, Artificial Sequence
  Syntetic construct
MRYILAVLLMVGMMAGAATAVTYEPTWESLDSRPNPAWFDEAKFGIFIHWGVYAVPAWGSKGKYSEWYWNDMMDPNGETWKFHLKTYGEDKFYQDFAPMFKAEMFDPAQWADIFARSGAKYVVLTSKHHEGFCLWPSPDSWNWNSVDIGPHRDLCGDLTQAVRDRGLKMGFYYSLYEWFNPIYKTDVHRYVDQHMLPQLKDLVNRYQPSLIFSDGEWDHPSDVWRSTEFLAWLYNESPSREDVIVDDRWGKDTRGHHGGYYTTEYGNIYQAPEDAFQKRKWEECRGMGASFGYNRNETIDEYKPAGELIHLLIELVARGGNLLLDIGPTADGRIPVIMQQRLLEIGDWLKENGEGIYGSSPWRVNAEGDSVRYTTRDGAVYAHLLKWPGAELALESPKAGGTVEASLLGWPEPLACKVENGKIHISMPVIPPDNNTIRHAFVIRLKGVE

SEQ ID NO: 2 - protein, 283 aa, Homo sapiens
MRAPGMRSRPAGPALLLLLLFLGAAESVRRAQPPRRYTPDWPSLDSRPLPAWFDEAKFGVFIHWGVFSVPAWGSEWFWWHWQGEGRPQYQRFMRDNYPPGFSYADFGPQFTARFFHPEEWADLFQAAGAKYVVLTTKHHEGFTNWPSPVSWNWNSKDVGPHRDLVGELGTALRKRNIRYGLYHSLLEWFHPLYLLDKKNGFKTQHFVSAKTMPELYDLVNSYKPDLIWSDGEWECPDTYWNSTNFLSWLYNDSPVKDEVVVNDRWGQNCSCHHGGYYNCEDKF

SEQ ID NO: 3 - protein, 285 aa, Macaca fascicularis
MRAPGERWRPAGAALWLLLLLLLLGATESVRRAQPLRRYTPDWPSLDSRPLPSWFDEAKFGVFIHWGVFSVPAWGSEWFWWNWQGEGRPQYQRFMRDNYPPGSSYADFGPQFTARFFHPEEWADLFQAAGAKYVVLTTKHHEGFTNWPSPVSWNWNSKDVGPHRDLVGELGTALRKRNIRYGLYHSLLEWFHPLYLLDKKNGFKTQYFVGAKTMPELYDLVNSYKPDLIWSDGEWECPDTYWNSTNFLSWLYNDSPVKDEVVVNDRWGQNCSCHHGGYYNCEDKF

SEQ ID NO: 4 - protein, 286 aa, Bos taurus
MRSWVVGARLLLLLQLVLVLGAVRLPPCTDPRHCTDPPRYTPDWPSLDSRPLPAWFDEAKFGVFVHWGVFSVPAWGSEWFWWHWQGEKLPQYESFMKENYPPDFSYADFGPRFTARFFNPDSWADLFKAAGAKYVVLTTKHHEGYTNWPSPVSWNWNSKDVGPHRDLVGELGTAIRKRNIRYGLYHSLLEWFHPLYLRDKKNGFKTQYFVNAKTMPELYDLVNRYKPDLIWSDGEWECPDTYWNSTDFLAWLYNDSPVKDEVVVNDRWGQNCSCHHGGYYNCKDKF

SEQ ID NO: 5 - protein, 283 aa, Canis familiaris
MKPWAVGLGPPPPAVPLLLLLLLGAALVRAAAPPRRYTPDWQSLDSRPLPDWFDKAKFGVFVHWGEFAVPAWGSEWFWWHWKGEGLPQYEQFMSENYPPGFSYADFGPQFTARFFHPDTWADLFQAAGARYVVLTTKHHEGFTNWPSSVSWNWNSNDVGPHRDLVGELGRALRKRNIRYGLYHSLLEWFHPLYLLDKKNNFKTQFFVRAKTMPELYDLVNRYEPDLIWSDGEWKCPDTYWNSTEFLSWLYNDSPVKDHVVVNDRWGQNCSCHHGGYYNCQDKY

SEQ ID NO: 6 - protein, 279 aa, Rattus norvegicus
MWDLKSEWWAVGFGLLLLLAASAQAGGLAPHHYTPDWPSLDSRPLPRWFDEAKFGLFVHWGVYSVPAWGSEWFWWHWQGEQSSAYVRFMKENYPPGFSYADFAPQFTARFFHPEEWADLFQAAGAKYVVLTAKHHEGFTNWPSAVSWNWNSKDVGPHRDLVGELGAAVRKRNIRYGLYHSLFEWFHPLYLLDKKNGLKTQHFVSTKTMPELYDLVNRYKPDLIWSDGEWECPDSYWNSTEFLAWLYNESPVKDQVVVNDRWGQNCSCRHGGYYNCEDKY

SEQ ID NO: 7 - protein, 269 aa, Mus musculus
MLLLLLLLLVAAAQAVALAPRRFTPDWQSLDSRPLPSWFDEAKFGVFVHWGVFSVPAWGSEWFWWHWQGDRMPAYQRFMTENYPPGFSYADFAPQFTARFFHPDQWAELFQAAGAKYVVLTTKHHEGFTNWPSPVSWNWNSKDVGPHRDLVGELGAAVRKRNIRYGLYHSLLEWFHPLYLLDKKNGFKTQHFVRAKTMPELYDLVNSYKPDLIWSDGEWECPDTYWNSTSFLAWLYNDSPVKDEVIVNDRWGQNCSCHHGGYYNCQDKY

SEQ ID NO: 8 - protein, 279 aa, Pongo pygmaeus
MRPQELPRLAFPLLLLLLLPPPPCPAHSATRFDPTWESLDARQLPAWFDQAKFGIFIHWGVFSVPSFGSEWFWWYWQKEKIPKYVEFMKDNYPPSFKYEDFGPLFTAKFFNANQWADIFQASGAKYIVLTSKHHKGFTLWGSEYSWNWNAIDEGPKRDIVKELEVAIRNRTDLRFGLYYSLFEWFHPLFLEDESSSFHKRQFPVSKTLPELYELVNNYQPEVLWSDGDGGAPDQYWNSTGFLAWLYNESPVRETVVTNDRWGAGSIYKHGGFYTCSDRY

SEQ ID NO: 9 - protein, 281 aa, Homo sapiens
MRPQELPRLAFPLLLLLLLLLPPPPCPAHSATRFDPTWESLDARQLPAWFDQAKFGIFIHWGVFSVPSFGSEWFWWYWQKEKIPKYVEFMKDNYPPSFKYEDFGPLFTAKFFNANQWADIFQASGAKYIVLTSKHHEGFTLWGSEYSWNWNAIDEGPKRDIVKELEVAIRNRTDLRFGLYYSLFEWFHPLFLEDESSSFHKRQFPVSKTLPELYELVNNYQPEVLWSDGDGGAPDQYWNSTGFLAWLYNESPVRGTVVTNDRWGAGSICKHGGFYTCSDRY

SEQ ID NO: 10 - protein, 273 aa, Rattus norvegicus
MRLGLLMFLPLLLLATRYRAVTALSYDPTWESLDRRPLPAWFDQAKFGIFIHWGVFSVPSFGSEWFWWYWQKERRPKFVDFMDNNYPPGFKYEDFGVLFTAKYFNANQWADLLQASGAKYVVLTSKHHEGFTLWGSAHSWNWNAVDEGPKRDIVKELEVAVRNRTDLHFGLYYSLFEWFHPLFLEDQSSAFQKQRFPVAKTLPELYELVTKYQPEVLWSDGDGGAPDHYWNSTDFLAWLYNESPVRDTVVTNDRWGAGSICKHGGYYTCSDRY

SEQ ID NO: 11 - protein, 275 aa, Mus musculus
MRLGFLMLLPLLLLPLLRPWGVTRALSYDPTWESLDRRPLPAWFDQAKFGIFIHWGVFSVPSFGSEWFWWYWQKEKKPQFVDFMNNNYAPGFKYEDFVVLFTAKYFNANQWADILQASGAKYVVFTSKHHEGFTMWGSDRSWNWNAVDEGPKRDIVKELEVAVRNRTGLHFGLYYSLFEWFHPLFLEDQSSSFQKQRFPVSKTLPELYELVNRYQPEVLWSDGDGGAPDHYWNSTGFLAWLYNESPVRKTVVTNDRWGVGSICKHGGYYTCSDRY

SEQ ID NO: 12 - protein, 269 aa, Unknown
  Omnitrophica
MRYILAVLLMVGMMAGAATAVTYEPTWESLDSRPNPAWFDEAKFGIFIHWGVYAVPAWGSKGKYSEWYWNDMMDPNGETWKFHLKTYGEDFKYQDFAPMFKAEMFDPAQWADIFARSGAKYVVLTSKHHEGFCLWPSPDSWNWNSVDIGPHRDLCGDLTQAVRDRGLKMGFYYSLYEWFNPIYKTDVHRYVDQHMLPQLKDLVNRYQPSLIFSDGEWDHPSDVWRSTEFLAWLYNESPSREDVIVDDRWGKDTRGHHGGYYTTEYGNIY